(19) United States
(12) Patent Application Publication
(10) Pub. No.: US 2009/0175916 A1
(43) Pub. Date: Jul. 9, 2009

(54) Sheet-shaped cosmetic product

(75) Inventors: Yoshiteru Kanomata, Fuji-shi (JP); Mitsunobu Yamamoto, Tokyo (JP); Shigeki Matsumura, Suita-shi (JP); Akiyoshi Takano, Fuji-shi (JP)

Correspondence Address: COHEN, PONTANI, LIEBERMAN & PAVANE LLP, 551 FIFTH AVENUE, SUITE 1210, NEW YORK, NY, 10176, US

(73) Assignee: Nippon Paper Papylia Co., Ltd., Shizuoka (JP)

(21) Appl. No.: 12/215,329

(22) Filed: Jun. 26, 2008

(30) Foreign Application Priority Data
Jun. 28, 2007 (JP) 2007-170459

(51) Int. Cl. A61K 8/02

(52) U.S. Cl. 424/401; 514/772.6; 514/772.3

(57) ABSTRACT

A sheet-shaped cosmetic product of the invention has a sheet and a stratified cosmetic cake disposed on the sheet with an adhesive layer sandwiched therebetween, and the adhesive layer and the cosmetic cake form a bonding zone in cooperation with each other, the bonding zone in which the adhesive layer and the cosmetic cake are engaged with and bonded to each other.

### BACKGROUND OF THE INVENTION

1. Field of the Invention

The present invention relates to a sheet-shaped cosmetic product having cosmetic cake with viscosity, which includes foundation, eye shadow, blusher, etc.

2. Description of the Related Art

The cosmetic cake is a compound of powdery cosmetic material that is a main component and viscous material. The viscous material solidifies the powdery cosmetic material into cosmetic cake and provides the cosmetic cake with desired viscosity.

In general, cosmetic products include containers made of metal, plastic or the like, in which the cosmetic cake is contained. Some of the cosmetic products are brought to users in the form of containers, and some in the form of sheets. A sheet-shaped cosmetic product of this type, for example, as disclosed in Unexamined Japanese Patent Application Publication No. 2005-40356 and Japanese Patent No. 3379086, includes a sheet-shaped base, a thin layer of cosmetic cake attached onto the base, and a transparent cover film covering the cosmetic cake. This cover film is detachable. The cosmetic cake of such a sheet-shaped cosmetic product is contained by an amount enough to be consumed in one to five times of making-up. The sheet-shaped cosmetic product is therefore suitable to be used as a sample and also suitable to be carried.

The base of the sheet-shaped cosmetic product is made, for example, of polyester. Adhesiveness between the base made of polyester and the cosmetic cake greatly depends upon the composition of the cosmetic cake. Accordingly, the cosmetic cake having a poor adhesion property with polyester is easily flaked off of the base. The easy flaking-off of the cosmetic cake causes the cosmetic cake to fall off of the base, and then drastically deteriorates the quality of the sheet-shaped cosmetic product. Furthermore, if the cosmetic cake is easily flaked off, this makes it difficult or impossible for the user to take up some of the cosmetic cake onto a sponge or a puff by rubbing the sponge or the puff against the cosmetic cake.

A possible way to solve the above problem is to increase the surface roughness of the base, for example, up to about 4 to 10 μm or to press the cosmetic cake against the base while heating the cosmetic cake. However, neither way can achieve sufficient adhesiveness between the base and the cosmetic cake, and prevent the flaking-off of the cosmetic cake from the base. As a matter of fact, the latter way might deteriorate the quality of the cosmetic cake by heating and pressing the cosmetic cake.

### SUMMARY OF THE INVENTION

It is an object of the present invention to provide a sheet-shaped cosmetic product that is capable of maintaining the quality of cosmetic cake and stably retaining the cosmetic cake on the base.

In order to accomplish the object, the sheet-shaped cosmetic product of the invention comprises a sheet, an adhesive layer formed on the sheet, cosmetic cake disposed on the adhesive layer, and a bonding zone formed of the cosmetic cake and the adhesive layer, the bonding zone in which the cosmetic cake and the adhesive layer are engaged with and bonded to each other.

More specifically, the cosmetic cake is a stratified compound containing powdery cosmetic material and viscous material.

The adhesive layer is made of adhesive having a swelling property with respect to liquid. In this case, the bonding zone is formed by swelling the adhesive on a contact surface of the adhesive layer, which comes into contact with the cosmetic cake. The bonding zone has a bumpy boundary surface engaged with the cosmetic cake. Preferably, the swelling of the adhesive is generated by liquid temporarily infiltrated into the cosmetic cake in a process of making the cosmetic cake. The cosmetic cake is formed by applying slurry of the cosmetic cake onto the adhesive layer. This slurry contains the liquid.

To be more concrete, the adhesive contains a synthetic resin swollen by alcohol serving as the liquid. The synthetic resin is either acrylic resin or polyurethane resin.

The sheet may include a mount for the cosmetic cake and a cover that is connected to the mount across a fold line and functions to cover the cosmetic cake.

Since the sheet-shaped cosmetic product of the invention comprises the bonding zone in which the cosmetic cake and the adhesive layer are engaged with and bonded to each other, the cosmetic cake adheres to the sheet through the adhesive layer with proper adhesive strength. In the bonding zone, the cosmetic cake and the adhesive layer are merely mechanically bonded together, so that the cosmetic cake is not damaged in quality. In other words, the cosmetic cake is stably fixed onto the sheet with the adhesive layer sandwiched therebetween while the quality of the cosmetic cake is maintained.

Other objects, advantages and the like of the invention will become clear in the Detailed Description of the Preferred Embodiments and drawings.

### BRIEF DESCRIPTION OF THE DRAWINGS

FIG. 1 is a perspective view schematically showing a sheet-shaped cosmetic product of one embodiment;

FIG. 2 is a sectional view showing a part of the cosmetic product shown in FIG. 1;

FIG. 3 is a flowchart showing a manufacturing process of the cosmetic product shown in FIG. 1; and

FIG. 4 is a view showing a part of FIG. 2 in an enlarged scale.

### DETAILED DESCRIPTION OF THE PREFERRED EMBODIMENTS

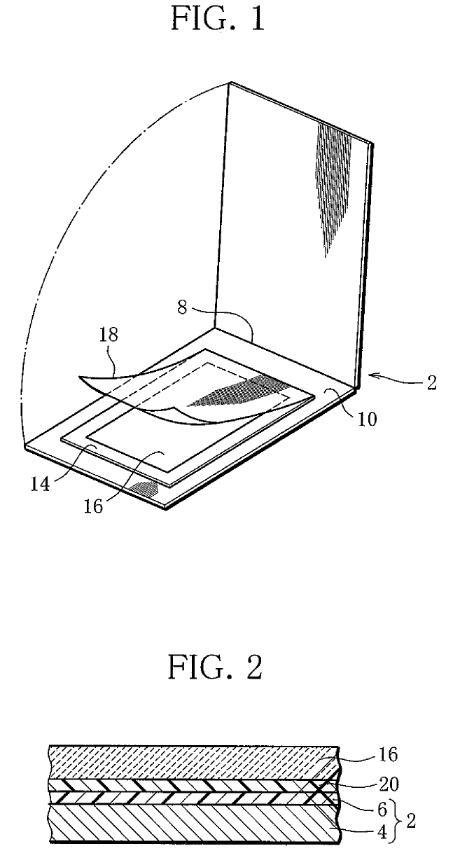

Referring to FIGS. 1 and 2, a sheet-shaped cosmetic product of one embodiment is provided with a sheet 2. The sheet 2 includes a cardboard 4 and a coating layer 6 laminated on the cardboard 4. The coating layer 6 is made, for example, of synthetic resin such as polyester. The sheet 2 may have a film affixed onto the cardboard 4, instead of having the coating layer 6. Alternatively, the sheet 2 may be made only of a film. The film in this case has proper thickness and is made of synthetic resin of the same quality as the coating layer 6.

The sheet 2 further has a fold line B. The fold line 8 divides the sheet 2 into a mount 10 and a cover 12. When the sheet 2 is folded along the fold line 8, the cover 12 is superimposed upon the mount 10. The folded sheet 2, namely the mount 10, is about the size of a business card.

A rectangular frame 14 is affixed onto the center of an upper surface of the mount 10. The frame 14 is made of paper and has given thickness. The frame 14 secures a rectangular recessed place in cooperation with the mount 10. In the frame 14, a stratified cosmetic cake 16, such as foundation, eye shadow and blusher, is maintained. The cosmetic cake is solid and yet has viscosity.

The cosmetic cake 16 is a compound of cosmetic material and viscous material. The viscous material provides viscosity to the cosmetic cake 16 and also solidifies the cosmetic cake 16. The cosmetic cake 16 is contained by an amount enough to be consumed in one to five times of making-up. The cosmetic cake 16 has a slightly smaller thickness than the frame 14.

The sheet-shaped cosmetic product further includes a transparent cover film 18 that covers the cosmetic cake 16. The cover film 18 is of the same size as the frame 14, and is detachably affixed onto an upper surface of the frame 14. According to the present embodiment, the cover film 18 includes an adhesive area in a portion of an inner surface, which comes into contact with the frame 14. That is to say, the adhesive area has a rectangular shape similar to that of the frame 14 and extends along an entire outer circumference of the cover film 18. The adhesive area may be formed in the upper surface of the frame 14, instead of the inner surface of the cover film 18. The adhesive area may also have re-adhesion property.

When the sheet-shaped cosmetic product is used, the user is able to expose the cosmetic cake 16 by peeling the cover film 18 off of the frame 14.

As illustrated in FIG. 2, the cosmetic cake 16 adheres to the coating layer 6 with an adhesive layer 20 sandwiched therebetween. The adhesive layer 20 is formed by applying adhesive onto the coating layer 6. The adhesive has such a property that it is swollen when infiltrated with liquid. As adhesive of this type, adhesive that chiefly contains acrylic resin or polyurethane resin is used. This adhesive, or an adhesive layer 20, has a swelling property with respect to alcohol (lower alcohol).

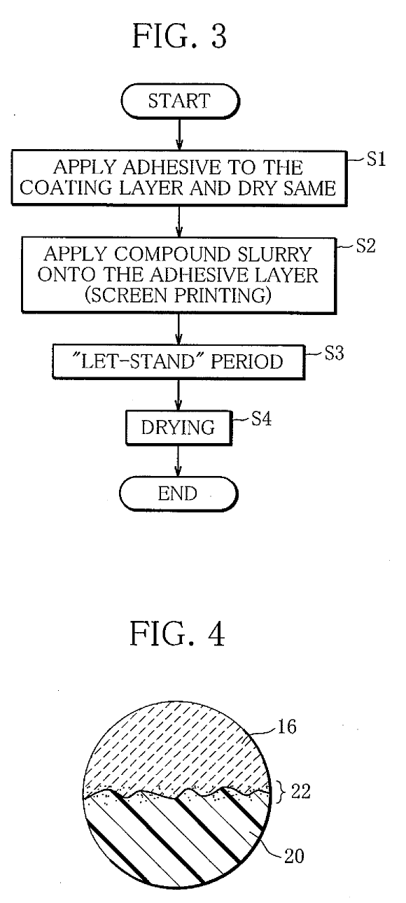

Due to the swelling property of the adhesive layer 20, the cosmetic cake 16 is formed on the adhesive layer 20 through a manufacturing process as shown in FIG. 3.

When the manufacturing process is carried out, slurry for making the cosmetic cake 16 is prepared. The slurry is produced by dispersing the compound into alcohol serving as solvent. After the slurry is prepared, the adhesive with the swelling property is applied onto the coating layer 6. The applied adhesive is then dried, to thereby form the adhesive layer 20 on the coating layer 6 (Step S1).

In the next stage, the slurry is applied onto the adhesive layer 20 by using a printing technique such as screen printing, to thereby form a slurry layer having given thickness (Step S2). The slurry layer is left for a given amount of time (Step S3). After the lapse of the “let-stand” period, the slurry layer is subjected to drying treatment (Step S4). The drying treatment emits the alcohol within the slurry layer. As a result, the solid cosmetic cake 16 made of a compound is formed on the adhesive layer 20.

While the slurry layer is left in Step S3, the alcohol contained in the slurry layer is infiltrated into the adhesive layer 20. The infiltrated alcohol swells a boundary surface of the adhesive layer 20, which is in contact with the slurry layer. The boundary surface then becomes bumpy. Therefore, convex portions of the boundary surface are stuck into the slurry layer, and at the same time, a portion of the slurry forming the slurry layer flows into concave portions of the boundary surface. In a boundary area in which the slurry layer and the adhesive layer 20 contact each other, there is a possibility that there is formed a mixed layer in which the slurry forming the slurry layer and the adhesive forming the adhesive layer 20 are mixed with each other. However, such a mixed layer is very thin and does not adversely affect the quality of the cosmetic cake 16.

When the alcohol within the slurry layer is emitted by the drying treatment, and the cosmetic cake 16 is formed, therefore, there is formed in between the cosmetic cake 16 and the adhesive layer 20 a mechanical bonding zone 22 in which the cosmetic cake 16 and the adhesive layer 20 are bonded together so as to be engaged with each other as illustrated in FIG. 4 with exaggeration. In a portion of the bonding zone 22, the compound forming the cosmetic cake 16 and the adhesive forming the adhesive layer 20 are mixed together.

Consequently, the cosmetic cake 16 firmly adheres to the adhesive layer 20 through the bonding zone 22. Since the bonding zone 22 is formed by the swelling of the adhesive layer 20, the cosmetic cake 16 is not adversely affected in quality, and is therefore reliably and stably fixed to the mount 10 with the adhesive layer 20 sandwiched therebetween.

### Embodiments

For the purpose of checking the adhesive strength of the cosmetic cake 16, the inventors prepared sheet-shaped base materials “A” and “B” serving as the coating layer 6, slurries “X” and “Y” for making cosmetic cake, adhesives “a” to “d” that are swollen by the slurries “X” and “Y”, and adhesives “e” and “f” that are not swollen by the slurries “X” and “Y”. Detailed descriptions of the base materials “A” and “B”, the slurries “X” and “Y”, and the adhesives “a” to “f” will be provided below.

Base Material “A”:

The base material “A” is made of polyester, and has a flat surface and a thickness of 25 μm.

Base Material “B”:

The base material “B” is made of polycarbonate (OPALUS EPC-E#150 (brand name) manufactured by Keiwa Inc.) and includes an embossed surface. The embossed surface has a surface roughness of 4.0 μm.

Slurry “X”:

The slurry “X” contains 60 percent by mass of foundation and 40 percent by mass of isopropyl alcohol (solvent). The foundation used here is a common one that was chosen among those available in the market.

Slurry “Y”:

The slurry “Y” contains 60 percent by mass of eye shadow and 40 percent by mass of isopropyl alcohol (solvent). The eye shadow used here is a common one that was chosen among those available in the market.

Adhesive “a”:

The adhesive “a” is acrylic emulsion (Seikaprene NF-303 (brand name) manufactured by Dainichiseika Color & Chemicals Mfg. Co., Ltd) which is commercially available. The acrylic emulsion contains thickener.

Adhesive “b”:

The adhesive “b” is urethane emulsion (Seikaprene UD-810 (brand name) manufactured by Dainichiseika Color & Chemicals Mfg. Co., Ltd) which is commercially available. The urethane emulsion contains thickener, too.

Adhesive “c”:

The adhesive “c” is a mixture containing 10 percent by mass of the acrylic emulsion, 4.0 percent by mass of carboxymethyl cellulose, 5.0 percent by mass of isopropyl alcohol, and 81.0 percent by mass of water. The acrylic emulsion has concentration corresponding to 45 percent by mass of a solid content.

Adhesive “d”:

The adhesive “d” is a mixture containing 10 percent by mass of the acrylic emulsion, 3.7 percent by mass of mineral-based thickener, 5.0 percent by mass of isopropyl alcohol, and 81.3 percent by mass of water. The acrylic emulsion has concentration corresponding to 45 percent by mass of a solid content.

Adhesive “e”:

The adhesive “e” is an aqueous solution containing 5 percent by mass of polyvinyl pyrrolidone (PVP) and simply has solubility with respect to alcohol.

Adhesive “f”:

The adhesive “f” is an aqueous solution containing 5 percent by mass of carboxymethyl cellulose (CMC). The CMC has no solubility with respect to alcohol, and is generally used solely as thickener.

In the next stage, sheet-shaped cosmetic products of Embodiments 1 to 9 and Comparative Examples 1, 2, 4 and 5 were fabricated according to the above-mentioned manufacturing process by using either the base material “A” or “B”, the slurry “X” or “Y”, and any one of the adhesives “a” to “f”. Meanwhile, sheet-shaped cosmetic products of Comparative Examples 3 and 6 were fabricated by using the base material “B” and either the slurry “X” or “Y” without using the adhesives.

Tables 1 and 2 show combinations of the base materials and the adhesives in Embodiments 1 to 6 and Comparative Examples 1 and 2 which use the slurry “X”, and combinations of the base materials and the adhesives in Embodiments 7 to 9 and Comparative Examples 4 and 5 which use the slurry “Y”, respectively.

TABLE 1
Slurry “X”

| | | | Application | Evaluation | | | | |
|---|---|---|---|---|---|---|---|---|
| | Base | | amount | Falling- | | | Total | |
| | material | Adhesive | (g/m²) | off | Attachment | Residue | evaluation | Comment |
| Embodiment 1 | B | a | 11.0 | Good | Good | Good | Good | Cosmetic cake |
| | | | | | | | | fell off a |
| | | | | | | | | little |
| Embodiment 2 | B | a | 20.0 | Good | Good | Good | Good | Good |
| Embodiment 3 | A | a | 3.0 | Good | Good | Good | Good | Good |
| Embodiment 4 | A | b | 3.0 | Good | Good | Good | Good | Good |
| Embodiment 5 | A | c | 1.2 | Good | Good | Good | Good | Good |
| Embodiment 6 | A | d | 1.5 | Good | Good | Good | Good | Good |
| Comparative | A | e | 1.0 | Good | Not good | Not good | Not good | Cosmetic cake |
| Example 1 | | | | | | | | stuck to the |
| | | | | | | | | base |
| Comparative | A | f | 1.0 | Not good | Not good | Good | Not good | Cosmetic cake |
| Example 2 | | | | | | | | fell off due to |
| | | | | | | | | impact |
| Comparative | B | None | 0.0 | Not good | Not good | Good | Not good | Cosmetic cake |
| Example 3 | | | | | | | | fell off due to |
| | | | | | | | | impact |

TABLE 2
Slurry “Y”

| | | | Application | Evaluation | | | | |
|---|---|---|---|---|---|---|---|---|
| | Base | | amount | Falling- | | | Total | |
| | material | Adhesive | (g/m²) | off | Attachment | Residue | evaluation | Comment |
| Embodiment 7 | A | a | 3.0 | Good | Good | Good | Good | Good |
| Embodiment 8 | A | b | 3.0 | Good | Good | Good | Good | Good |
| Embodiment 9 | A | c | 1.2 | Good | Good | Good | Good | Good |
| Comparative | A | e | 1.0 | Good | Not good | Not good | Not good | Cosmetic cake |
| Example 4 | | | | | | | | stuck to the |
| | | | | | | | | base |
| Comparative | A | f | 1.0 | Not good | Not good | Good | Not good | Cosmetic cake |
| Example 5 | | | | | | | | fell off due |
| | | | | | | | | to impact |
| Comparative | B | None | 0.0 | Not good | Not good | Good | Not good | Cosmetic cake |
| Example 6 | | | | | | | | fell off due |
| | | | | | | | | to impact |

Specific manufacturing processes for manufacturing Embodiments 1 to 9 and Comparative Examples 1, 2, 4 and 5 will be described below.

The adhesive was applied onto the base material by screen printing. For the screen printing, a printing plate having 200 mesh and a film thickness of 30 μm was used.

The slurry was applied onto a dried adhesive layer by carrying out the screen printing three times. For the screen printing, a printing plate having 60 mesh and a film thickness of 150 μm was used.

A “let-stand” period to be taken after the application of the slurry is 20 minutes at most, but preferably about 0 to 15 minutes although depending upon the kinds of the adhesives and the compositions of the slurries.

The slurry was dried by applying infrared rays onto and/or blowing warm air onto the applied slurry, thereby heating the slurry up to 100° C. This heating state is maintained for about one minute. The drying treatment of the slurry sufficiently emits the alcohol (solvent) contained in the slurry, and stops the swelling of the adhesive layer, or of the adhesive.

The manufacturing processes of Comparative Examples 3 and 6 are the same as those of Embodiments 1 to 9 and Comparative Examples 1, 2, 4 and 5, except that the application of the adhesive is omitted in Comparative Examples 3 and 6.

The sheet-shaped cosmetic products of Embodiments 1 to 9 and Comparative Examples 1 to 6 do not have the cardboard 4 and the frame 14 that are included in the sheet-shaped cosmetic product shown FIG. 1.

Tables 1 and 2 also show evaluation results of Embodiments 1 to 9 and Comparative Examples 1 to 6 as well. The evaluation results relate to “Falling-off Evaluation,” “Attachment Evaluation,” “Residue Evaluation” and “Total Evaluation.”

“Falling-off Evaluation” was performed by the steps of affixing a cardboard onto a surface of the cosmetic cake of the sheet-shaped cosmetic product with double-sided tape, applying a given impact to the cosmetic cake through the cardboard, and checking if the cosmetic cake falls off of a base material due to the impact. More specifically, the impact on the cosmetic cake was given by an inspector's flicking an outer surface of the cardboard.

If the cosmetic cake did not fall off, it was determined that the sheet-shaped cosmetic product was good. If the cosmetic cake fell off a lot, it was determined that the sheet-shaped cosmetic product was a defective that was not suitable for use.

“Attachment Evaluation” was performed by the steps of rubbing a surface of the cosmetic cake with a sponge for foundation five times, and checking how much of a compound forming the cosmetic cake is attached to the sponge. If the compound was sufficiently attached to the sponge, it was determined that the sheet-shaped cosmetic product was good. If the compound was hardly attached to the sponge, it was determined that the sheet-shaped cosmetic product was defective.

“Residue Evaluation” was performed by the steps of wiping off the cosmetic cake with a sponge for foundation in order to remove the cosmetic cake 16 from a base material, and checking an amount of the cosmetic cake remaining on the base material. If the cosmetic cake hardly remained on the base material, it was determined that the sheet-shaped cosmetic product was good. If much of the cosmetic cake remained, it was determined that the sheet-shaped cosmetic product was defective.

“Total Evaluation” determined that the sheet-shaped cosmetic product was good if the results of “Falling-off Evaluation,” “Attachment Evaluation” and “Residue Evaluation” were all good, and determined that the sheet-shaped cosmetic product was defective if any one of the results of “Falling-off Evaluation,” “Attachment Evaluation” and “Residue Evaluation” was defective.

The cosmetic cake of Comparative Examples 3 and 6 was not attached onto the base material with the adhesive layer interposed therebetween but directly attached onto the base material due to its own adhesive force. Even if a surface of the base material was formed into an embossed surface, adhesiveness between the base material and the cosmetic cake 16 was low. The cosmetic cake therefore fell off of the base material on small impact.

The cosmetic cake of Comparative Examples 1 and 4 was attached onto the base material through the adhesive layer (adhesive “e”) having solubility in alcohol. For this reason, an adhesive force between the cosmetic cake and the base material was great, and the cosmetic cake was reliably prevented from falling off of the base material. In this case, however, the adhesive “e” dissolved by alcohol deeply permeates the cosmetic cake, and a mixed area of the cosmetic cake and the adhesive “e” is too large. It is then difficult to rub off the compound from the cosmetic cake by using the sponge.

The cosmetic cake of Comparative Examples 2 and 5 was attached onto the base material through the adhesive layer (adhesive “f”). The adhesive “f” is used exclusively as thickener and is not soluble in alcohol. Therefore, adhesive strength between the cosmetic cake and the adhesive layer was small. The cosmetic cake easily fell off of the base material, and it was difficult to rub off the compound from the cosmetic cake.

As opposed to Comparative Examples 1 to 6, the cosmetic cake of Embodiments 1 to 9 was attached onto the base material with the adhesive layer (one of the adhesives “a” to “d”) interposed therebetween. The bonding zone 22 (see FIG. 4) was located in between the cosmetic cake and the adhesive layer. Accordingly, the cosmetic cake was well attached onto the base material and was then prevented from falling off of the base material. The adhesion property of the cosmetic cake is not affected by the surface roughness of the base materials “A” and “B”.

As is apparent from Tables 1 and 2, the cosmetic cake of Embodiments 1 to 9 is good not only in “Falling-off Evaluation” but also in “Attachment Evaluation” and “Residue Evaluation”, and thus has high quality.

When the cosmetic cake was fabricated in the same manner by using different brands of foundations and eye shadows to be used for making the slurries “X” and “Y”, the evaluation results of the cosmetic cake were almost identical to those of the cosmetic cake of Embodiments 1 to 9.

As is clear from the above explanation, each of the sheet-shape products of Embodiments 1 to 9 has the bonding zone between the cosmetic cake and the adhesive layer. Therefore, the adhesive strength between the cosmetic cake and the adhesive layer is properly secured. Since the adhesive layer is firmly attached to the base material, namely the coating layer of the sheet, the cosmetic cake is not damaged in quality and is well maintained on the sheet through the adhesive layer.

Although Embodiments 1 to 9 provide the sheet-shaped cosmetic products for foundation and eye shadow, the present invention is applicable to a sheet-shaped cosmetic product for blusher.

It is possible to use, as an adhesive having the swelling property with respect to liquid, acrylic resin or polyurethane resin having the swelling property with respect to acetone. In this case, acetone is used as solvent for making slurry.

The slurry can be dried by various drying methods without using infrared rays or hot air.

The invention may be modified in various ways without deviating from the concept thereof.

What is claimed is:

1. A sheet-shaped cosmetic product comprising:
a sheet;
an adhesive layer formed on said sheet;
cosmetic cake disposed on said adhesive layer; and
a bonding zone formed of said cosmetic cake and said adhesive layer, said bonding zone in which said cosmetic cake and said adhesive layer are engaged with and bonded to each other.

2. The cosmetic product according to claim 1, wherein:
said cosmetic cake is a stratified compound containing powdery cosmetic material and viscous material.

3. The cosmetic product according to claim 1, wherein:
said adhesive layer is made of adhesive having a swelling property with respect to liquid; and
said bonding zone is formed by swelling the adhesive on a contact surface of said adhesive layer, which comes into contact with said cosmetic cake, and has a bumpy boundary surface engaged with said cosmetic cake.

4. The cosmetic product according to claim 3, wherein:
the swelling of the adhesive is generated by the liquid temporarily infiltrated into said cosmetic cake in a process of making said cosmetic cake.

5. The cosmetic product according to claim 4, wherein:
said cosmetic cake is formed by applying slurry of said cosmetic cake onto said adhesive layer, the slurry containing the liquid.

6. The cosmetic product according to claim 3, wherein:
the adhesive contains a synthetic resin swollen by alcohol serving as the liquid.

7. The cosmetic product according to claim 6, wherein:
the synthetic resin is either one of acrylic resin and polyurethane resin.

8. The cosmetic product according to claim 1, wherein:
said sheet includes a mount for the cosmetic cake and a cover connected to the mount across a fold line, the cover functioning to cover said cosmetic cake.
